(19) United States
(12) Patent Application Publication
(10) Pub. No.: US 2006/0003376 A1
(43) Pub. Date: Jan. 5, 2006

(54) Non-destructive quality control method for microarray substrate coatings via labeled doping

(76) Inventors: Samuel D. Conzone, Mainz (DE); Luis A. Burzio, Mountain Top, PA (US); Dan Haines, Lake Ariel, PA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/213,984

(22) Filed: Aug. 30, 2005

Related U.S. Application Data
(62) Division of application No. 10/600,347, filed on Jun. 23, 2003.

(51) Int. Cl. C12Q 1/68; C12M 1/34; C03C 3/089

(52) U.S. Cl. 435/6; 435/287.2; 501/65

(57) ABSTRACT

A non-destructive method of determining the uniformity of a coating on a substrate comprises coating the substrate with a homogenous coating mixture comprising a labeled compound and a chemically functional compound to which other chemical moieties can be bound, and detecting the presence and locations of the labeled compound on the surface. The invention also relates to a surface for attachment of molecules comprising a substrate and a coating thereon of a homogeneous mixture of a labeled compound and a first chemically functional compound to which other chemical moieties can be bound.

### BACKGROUND

Coatings on substrates for use in supporting high-density biological or chemical arrays are often based on monolayers, multilayers or hydrogels. Monolayers and multilayers are typically very difficult to characterize due to their very thin nature (i.e., typically <5 nm). Hydrogels are typically >5 nm thick, but are also difficult to characterize. The characterization of these coatings on glass is further complicated by the fact that they exhibit refractive indices that are very similar to glass. This similarity in refractive indices renders the coatings undetectable to most optical inspection and spectroscopic techniques.

In a typical microarray, biological probes (DNA, protein, carbohydrate, cells) are immobilized on a coated substrate in an arrayed format. An array can contain anywhere from tens to tens of thousands of probes per cm². Further, each of these biological probes plays an important role in the microarraying assay. This assembly of immobilized biological probes comprises the microarray. The microarray is then used to characterize an unknown sample of biological targets in a multiplexed manner. The biological targets (e.g., RNA as obtained from a DNA expression experiment) are typically isolated, tagged with a fluorescent dye and then introduced to the coated microarray surface. Once fluorescently tagged targets are introduced to the microarray surface, hybridization reactions can occur between complimentary probes and targets. After biological conjugation (hybridization) is complete, the microarray is typically placed in a laser scanner to identify which probe/target conjugates (hybrids) were formed. Each biological probe must be bound to the coated substrate in a uniform manner to ensure the integrity of the resulting microarray. Nonuniform substrate coatings will result in nonuniform probe immobilization and the resulting microarray will not be an accurate assay tool.

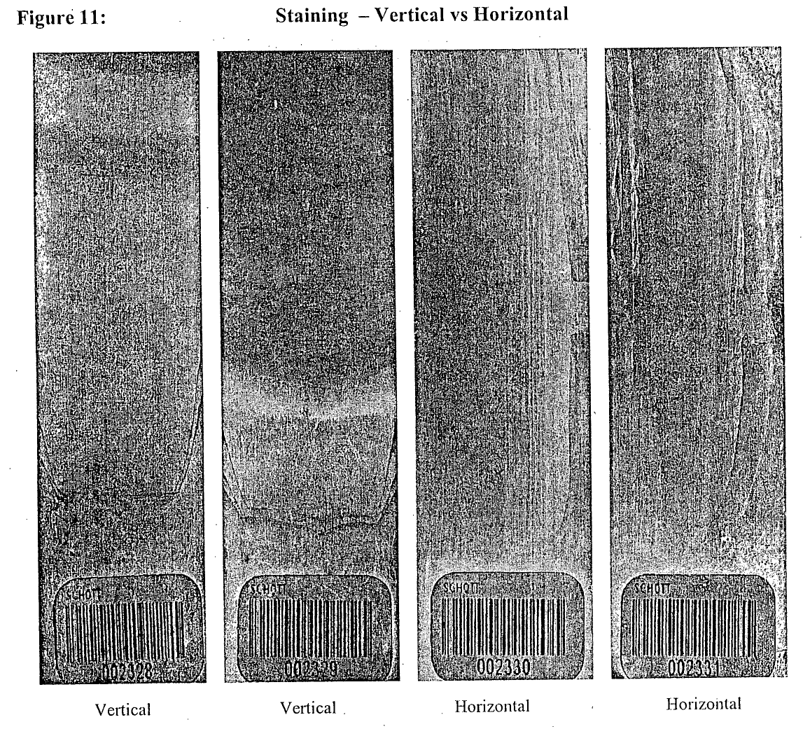

Traditional techniques for assessing quality and uniformity of a coated substrate often assess only a small portion of a coating on a substrate and in many cases destroy the substrate making it unusable for further assay activity. Even contact angle measurements, whereby a droplet of water is placed on the surface of a silane-coated substrate to characterize the hydrophobicity or hydrophilicity of the surface coating, is generally considered a destructive method for quality control (QC). Furthermore, only a very small percentage of the entire coated glass surface is tested (typically <1%). Similarly, DNA hybridization testing, whereby nucleic acid (e.g., oligonucleotide or cDNA) probes are arrayed and immobilized onto a coated glass substrate; is also a destructive method, although <1% of the coated surface is characterized. The nucleic acid probes are hybridized with fluorescently tagged complimentary targets and scanned, e.g., by an Axon scanner and signal to background (S/Bk) ratios can be calculated, thereby characterizing the biological performance of the coated substrate. Finally, chemical staining, whereby a coated substrate is stained with a fluorescent dye molecule that has an affinity for functional groups on the surface, is both a destructive quality control technique and a secondary or indirect means of analysis. The resulting stained, coated substrates are characterized using a commercially available scanner, and the fluorescence (e.g., Cy-3) is typically correlated with coating content/consistency on the surface (i.e., amine). It is often difficult to determine whether coating nonuniformities are caused by the primary substrate coating or the secondary staining process (See FIG. 11). Most spectroscopic techniques available for investigating coatings on substrate surfaces are based on the use of electrons or x-rays for imaging or chemical analysis. X-ray photoelectron spectroscopy (XPS) is a surface characterization technique whereby x-ray radiation is used to liberate core electrons from atoms within the coating on a substrate surface. The liberated electrons are called photoelectrons. The energy of the resulting photoelectrons is dependent upon the energy of the incident x-ray radiation, less the binding energy of the core electron. Thus, the photoelectron energy spectrum can be used to deduce information about the chemical composition, concentration and ligand field state of atoms within a coating. Atomic Force Microscopy (AFM) is a surface characterization technique whereby a “nano-stylus” is used to probe the roughness of a surface using piezoelectric-based detection of the stylus deflections. Alternatively, a Scanning Electron Microscopy (SEM) can be used to view the surface of a coated substrate using backscattered and/or secondary electrons.

While there are numerous surface characterization methods and techniques available that are both destructive and non-destructive (e.g., FTIR, XPS (x-ray photoelectron spectroscopy), ellipsometry, AFM (atomic force microscopy, IR (infrared spectroscopy), SPR (surface plasmon resonance), QCM (quartz crystal microbalance), and SAW (surface acoustic wave)), these methods and techniques are generally prohibitively expensive for quality control applications. Such methods and techniques are fully conventional. See for example, Popat, et al., Surf. Coat. Technology 2002, 154, 253-261; Chirakul et al., Langmuir 2002, 18, 4324-4330; Vallant et al., J. Phys. Chem. 1998, 102,7190-7197; Wasserman et al., Langmuir 1989, 5, 1074-1087; Ostuni et al., Coll. Surf. B. 1999, 15, 3-30; Tompkins et al., Spectroscopic Ellipsometry and Reflectometry: A User's Guide 1999, Wiley-Interscience; Abraham Ulman, Characterization of Organic Thin Films 1994; Shaun Wilson (Ed) et al., and Encylcopedia of Materials Characterization: Surfaces, Interfaces, Thin Films 1992, Butterworth-Heinemann.

These traditional coating characterization techniques (with the exception of staining) only characterize a very small percentage of the surface (typically <1%). Thus, they are not good indicators of overall coating uniformity. Moreover, these techniques are often destructive, meaning they can only be used to indirectly assess the overall quality of a batch. Typically, only a few slides in a batch will be tested and their overall coating uniformity is used as an indicator of the coating uniformity of the overall batch. Once a coated substrate has been tested by a destructive technique it is no longer suitable for assay use because the binding properties of the chemically functional coating compound have been modified or destroyed. While the staining process can characterize the entire coating of a substrate, it is a destructive technique that is fraught with process related artifacts resulting from the direction that the dip-staining process was executed (see FIG. 11).

### SUMMARY OF THE INVENTION

The present invention relates generally to surfaces useful in a non-destructive method of determining the uniformity of a coating on a substrate, particularly substrates which are suitable for the attachment of biomolecules such as carbohydrates, nucleic acids, including oligonucleotides, proteins and peptides, as well as cells and tissues. The invention also relates to fluorescently labeled substrates for attaching biomolecules. Fluorescently labeled compounds provide detectability to the substrate coating without deleteriously affecting the biological performance of the primary functional coating. The fluorescently labeled substrates will not interfere with further assay activity because the fluorescent labeled compounds are chosen so that they do not fluoresce in the spectral regions typically used for microarraying applications. Further, the fluorescent doping occurs at a low level that does not affect the biological performance of the primary chemically functional compound.

The present invention relates to a non-destructive method of determining the uniformity of a coating on a substrate and to labeled substrates for covalent attachment of biomolecules. Preferably, the label is a fluorescent one. The substrate is coated with a homogeneous mixture of a fluorescent-labeled compound (minor coating constituent) and a chemically functional compound (major coating constituent) to which other chemical moieties (e.g., biological probes and biomolecules) can be bound during microarraying experiments. Such a coating mixture allows both the primary functional compound and the fluorescent-labeled compound to be coated onto a substrate simultaneously. The fluorescent-labeled compound acts as a dopant, imparts detectability to the coated substrate surface and can be non-destructively determined by label detection and localization, e.g., for fluorescence, by exposure to energy in the 200-700 nm wavelength regions. The fluorescently labeled compound fluoresces in a region that will not interfere with subsequent fluorescent characterization of attached biomolecules during a microarray experiment and will not cause high background fluorescence during bioarray experiments.

Although this application discusses a preferred aspect of the invention, i.e., fluorescent-labeling, other labels are also included, such as, for example, nuclear labels (e.g., Tc), NMR labels (e.g., Gd⁺³), etc., as long as they can be detected and localized.

The homogeneous mixture of a fluorescent-labeled compound and a chemically functional compound is preferably applied to the substrate by chemical vapor deposition, sputtering, dip coating, spin coating, ion beam deposition, flame hydrolysis deposition, laser pyrolysis deposition, liquid phase deposition, electron beam deposition, plasma arc deposition or evaporation deposition, but other techniques can be used.

The invention also relates to a substrate surface for attachment of biomolecules comprising: a coating of a homogeneous mixture of a fluorescent-labeled compound and a chemically functional compound to which other chemical moieties can be bound. The dopant portion of the homogenous coating will not interfere with the attachment of other moieties such as biomolecules.

DNA, modified or unmodified nucleic acids, antibodies, antigens, proteins, oligonucleotides, carbohydrates, sugars or any biomolecules (e.g., tissues or cells) can be attached to the coated substrates of the invention. Although a variety of substrates are contemplated, as long as compatible with the end use such as a bioassay, a preferred substrate is glass and preferably a gold or other coated glass or a low self-fluorescent glass, for example borosilicate or soda lime silicate glass among others. The chemically functional compound to which other moieties (e.g., biomolecules) can be attached is preferably a functionalized alkoxysilane, chlorosilane, hydrogel or functionalized alkanethiol. When the chosen substrate is gold coated, the preferred functional compound is a functionalized alkanethiol. If the chosen substrate is glass the preferred functional compound is a hydrogel, chlorosilane or an alkoxysilane and the most preferable functional compound is a multiaminoorganosilane such as N-(2 -aminoethyl)-3-aminopropyltrimethoxysilane (EDA), trimethoxysilyipropyl-diethylenetriamine (DETA) or (aminoethyl aminomethyl) phenethyltrimethoxysilane (PEDA).

A preferred use of the present invention is to covalently or non-covalently immobilize a controlled density of biomolecules, preferably nucleic acid molecules and particularly nucleic acid oligomers, onto the coated substrate. The present invention thus can provide sensors, biosurfaces or biomaterials for a variety of biological, analytical, electrical or optical uses. The coated substrates can also be used as “adhesive scaffolds” upon which cell and tissue engineering can be conducted.

Thus, in general, the coated substrates of the present invention can be used in processes for detecting and/or assaying a molecule, e.g., with biological activity in a sample. When a coated substrate as described above is used and such a sample molecule becomes attached, the detection or assay can be carried out using a reagent, fluorescent or otherwise, which detects the presence of the attached molecule.

The fluorescently labeled substrates of the present invention provide fluorescence in a region that does not interfere with further assay activity because the fluorescently labeled compounds preferably do not fluoresce in the spectral regions typically used for DNA or protein arraying applications. Further, the doping occurs at a low level that does not affect the biological performance of the primary chemically functional compound. After coating, the fluorescent-doped coating can be characterized in a light box using UV, visible or IR radiation as an excitation source and a suitable UV, visible or IR based detection system. The coating may also be characterized in a fluorimeter. The resulting fluorescence of the doped coating can be used to characterize coating uniformity in a non-destructive manner.

This invention thus enables non-destructive assessment of the quality (e.g., uniformity and homogeneity) of a chemically functional substrate coating, despite thicknesses of often less than 5 nm and the fact that the undoped coating is nearly undetectable (e.g., no absorption is visible and its refractive index is essentially the same as that of the glass).

A more complete appreciation of the invention will be readily obtained by reference to the accompanying drawings, wherein:

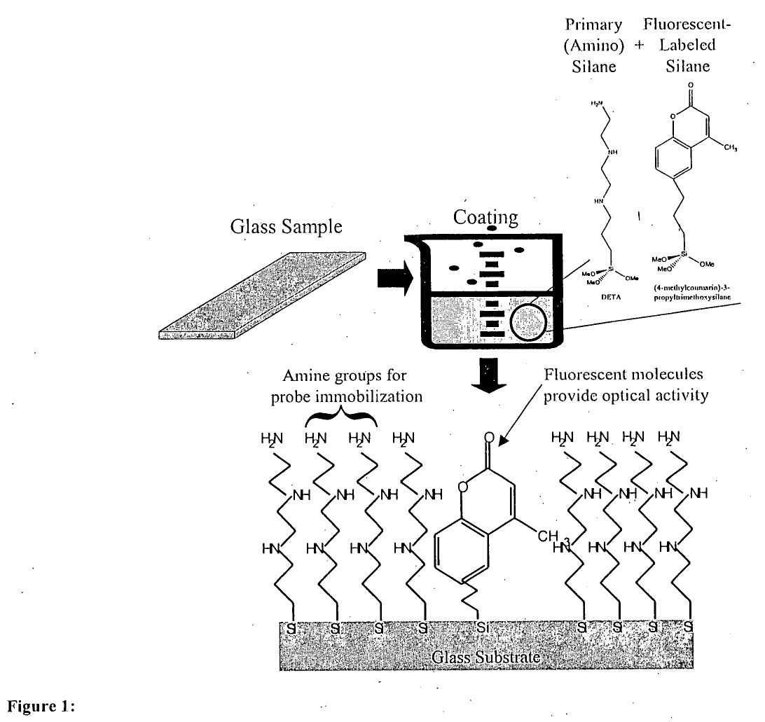

FIG. 1—is a pictorial representation showing how primary and fluorescent-labeled silane molecules can be coated onto a glass surface to (1) provide chemical functionality needed for probe immobilization during a microarraying experiment and (2) provide fluorescent optical activity that can be used to characterize coating uniformity.

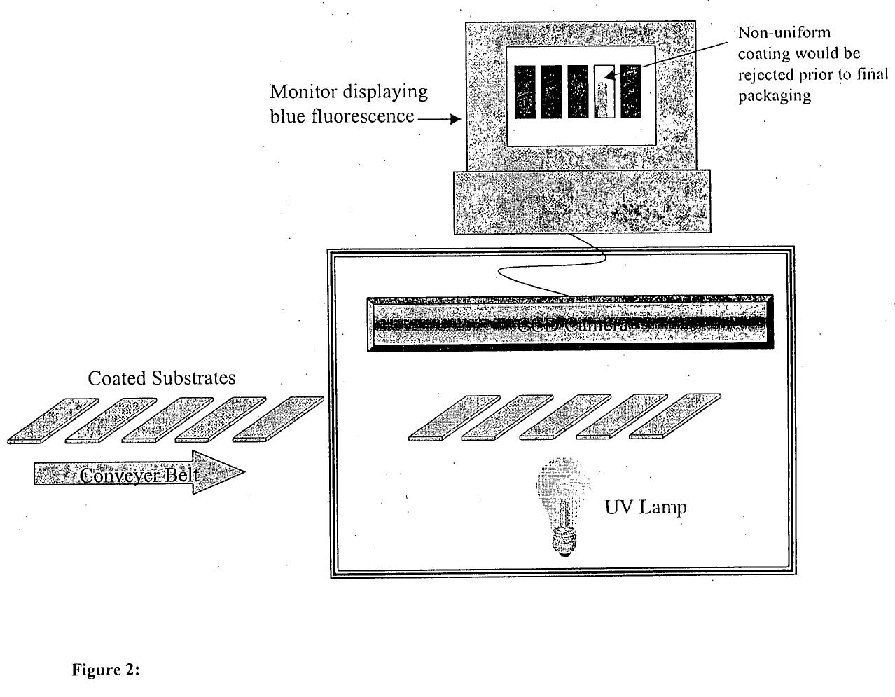

FIG. 2—is a pictorial representation of an automated quality control system, whereby UV light is used to illuminate the “mixed” coated substrates. Blue fluorescence is captured by a CCD camera and then displayed on a monitor. Machine vision and/or a human operator could remove substrates with non-uniform fluorescence.

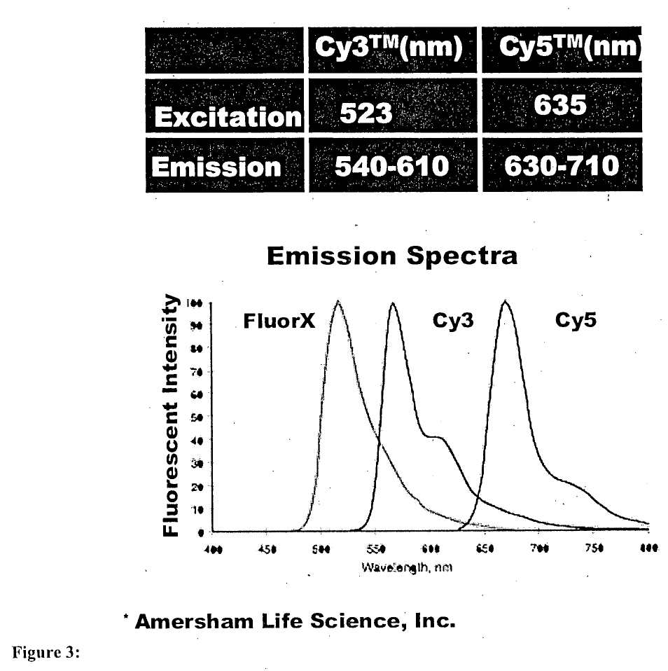

FIG. 3—shows excitation and emission data for Fluor X™, Cy3™, and Cy5™ chemical dyes, which are commonly used for DNA and protein arraying applications. Fluorescent-labeled coating molecules are chosen such that they do not fluoresce under the same excitation/emission conditions as the dyes used during a particular microarraying experiment.

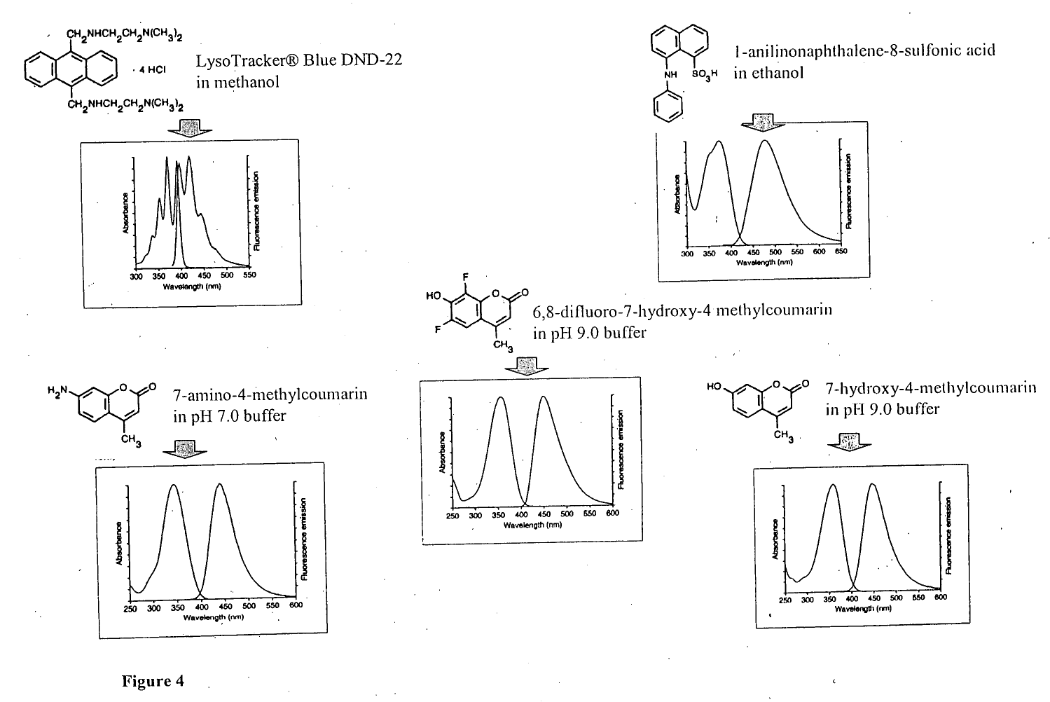

FIG. 4—shows chemical structure, absorbance and fluorescent emission spectra for five “blue” chemical dyes that absorb in the UV and have emission maxima below 500 nm. Such dyes exhibit fluorescence when illuminated with a UV light, but do not interfere with the fluorescent tags (i.e., Cy3 or Cy5) that are typically used during a microarray experiment. This list of chemical dyes is non-exhaustive.

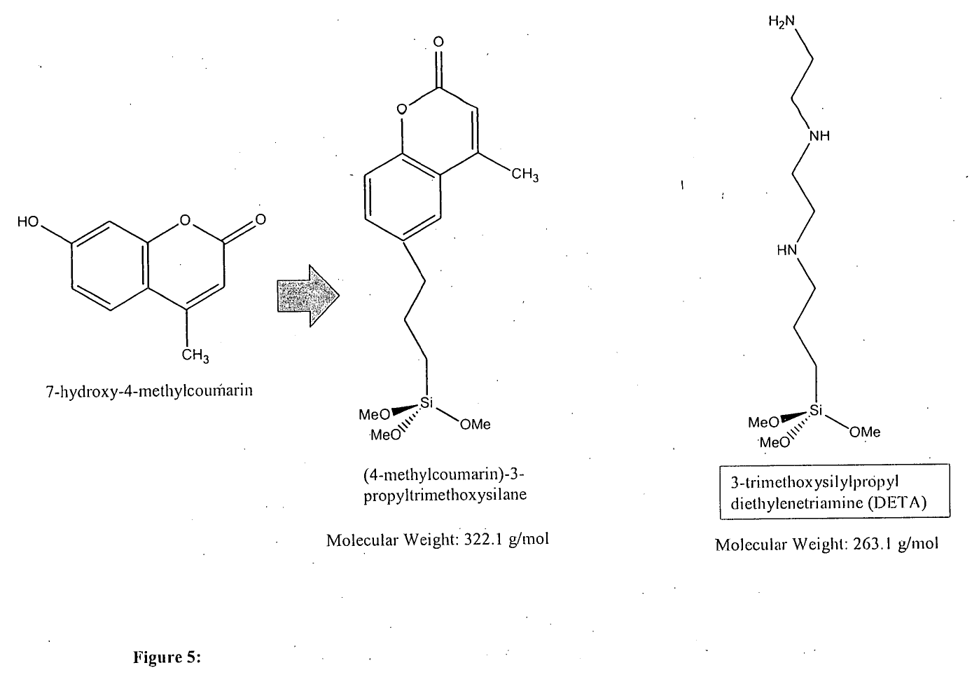

FIG. 5—is a pictorial representation of how a “blue” chemical dye (left) can be converted into a silane, which can subsequently be coated onto a glass surface. The resulting (4-methylcoumarin)-3-propyltrimethoxy silane is approximately the same size as DETA (a primary amino-silane molecule that can be coated onto a glass and used for nucleic acid immobilization for DNA microarray applications).

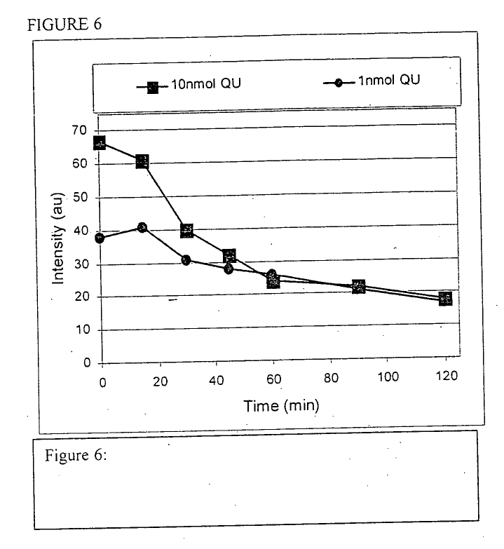

FIG. 6 is a chart representation depicting the decreasing intensity of the fluorescent signal of QU coated slides at various time during UV exposure. Slides were exposed to 365 nm light and the fluorescence was measured at different times. The typical intensity for uncoated glass in this case is 17. At 60 minutes the fluorescent signal of the QU coated slides is the same as for the uncoated glass.

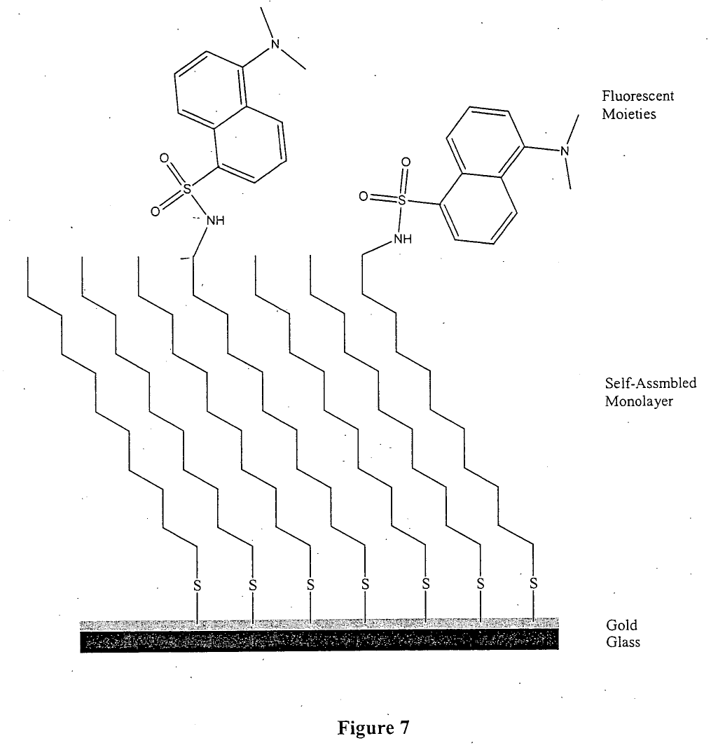

FIG. 7 is a pictorial representation of a gold-coated glass substrate with a self-assembled monolayer having thiol (S) head groups attached covalently to a gold surface, alkyl (CH₂) groups whose interaction with one another gives ordering to the monolayer, and random fluorescent (i.e., dansyl amide) tail groups.

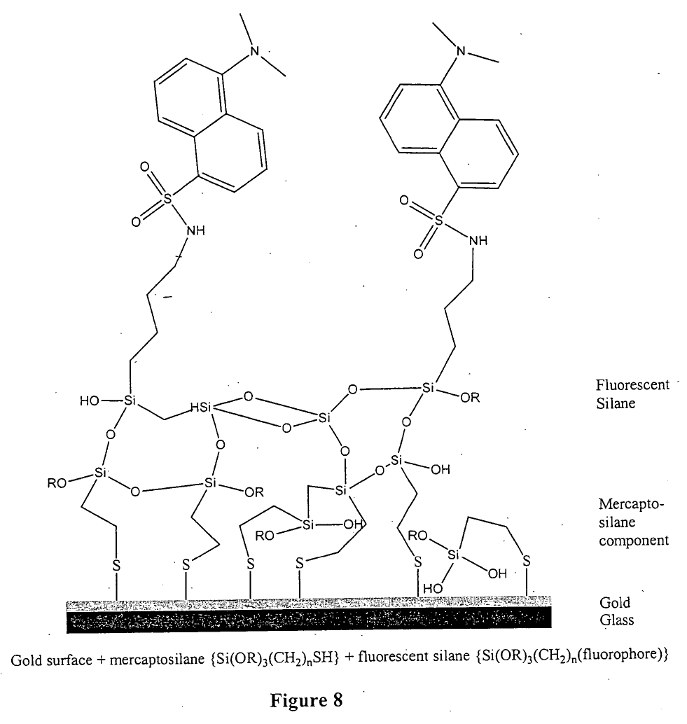

FIG. 8 is a pictorial representation of the reaction of a gold-coated glass substrate with a mercaptosilane, resulting in either a disordered surface (for short mercapto-silanes as shown) or an ordered surface for long mercapto-silanes. Silanes can crosslink with one another, shown by Si—O—Si bonds. The derivatized surface can further react with a fluorescent second silane (i.e., a dansyl amide), covalently bonding through Si—O—Si bonds.

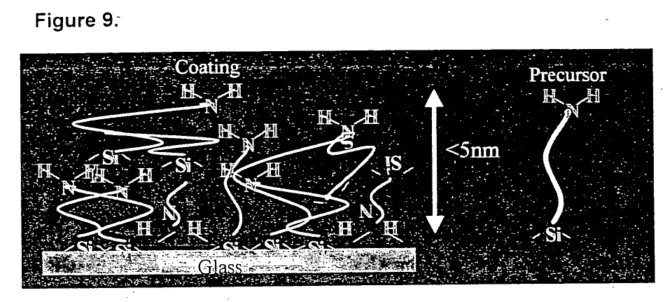

FIG. 9 is a pictorial representation of a monolayer coating on a glass surface.

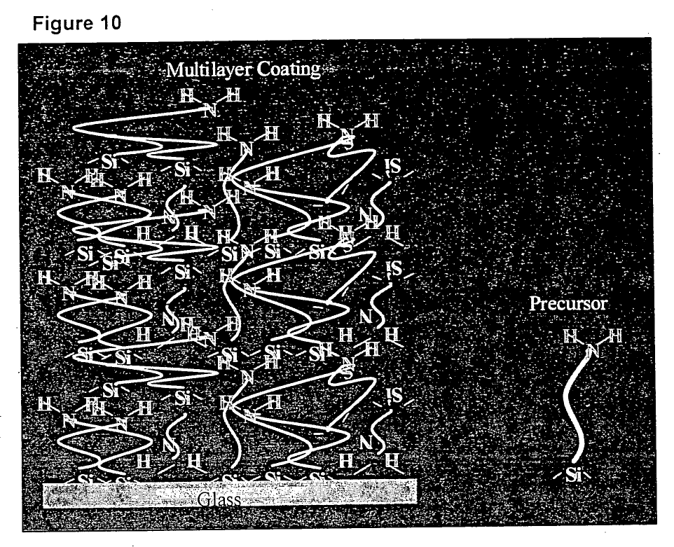

FIG. 10 is a pictorial representation of a multilayer coating on a glass surface depicting a multiple layer coating of the multiamino precursor.

FIG. 11 is a photograph showing process related artifacts resulting from the direction that the dip-staining process was executed. The staining artifacts (streaks) are dependent upon the direction of dipping during the staining experiment.

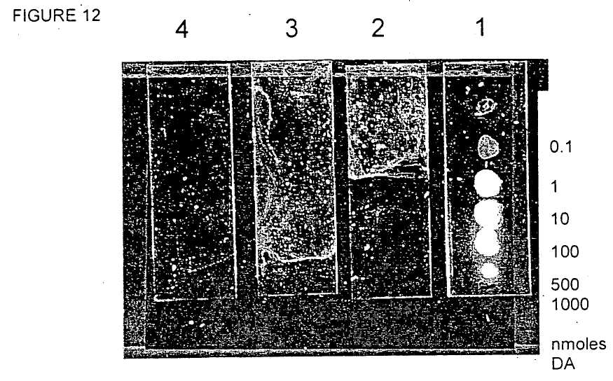

FIG. 12 is a photograph showing various concentrations of dansylamide coatings on glass slides. Slide 1 shows 1 μl drops containing different amounts of dansylamide. Slides 2 and 3 show 10 nmoles of dansylamide spread across the slide manually. Slide 4 shows 0.1 nmol dansylamide spread across the slide.

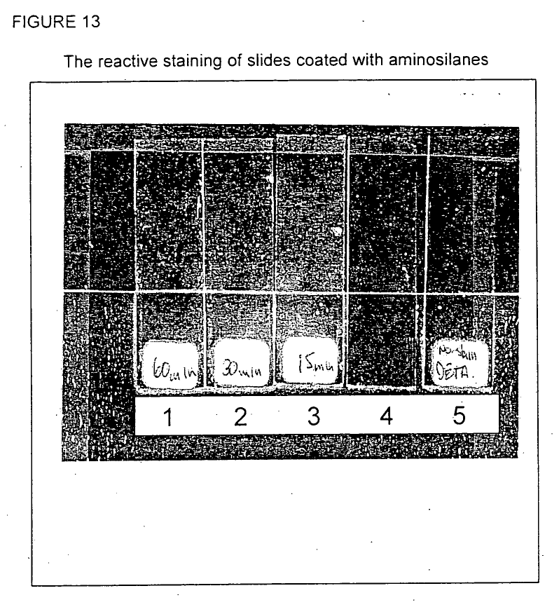

FIG. 13 is a photograph showing aminosilane coated slides incubated with Fluorescamine. Slides 1, 2, and 3 were incubated for 60, 30, and 15 minutes respectively. Slide 4 is an uncoated control that was incubated for 60 minutes. Slide 5 is an untreated DETA coated slide.

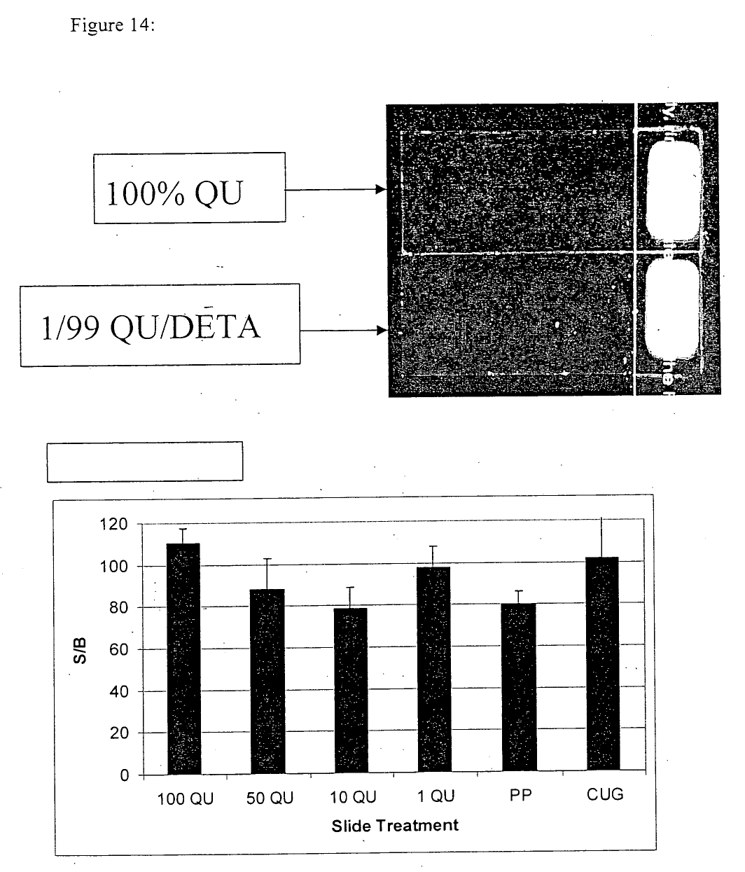

FIG. 14 depicts clean slides that were dip coated in solutions containing various concentrations of a fluorescent silane (N-triethoxysilylpropylquinineurethane-QU) and a non-fluorescent silane (3-trimethoxysilylpropyl diethylenetriamine-DETA). The top figure depicts the fluorescent differences between slides coated with only QU (100% QU) and a slide coated with 1% QU and 99% DETA (1/99% QU/DETA). The bottom graph depicts the hybridization results in terms of signal to background ratios for various concentrations of QU as well as slides without QU (PP and CUG).

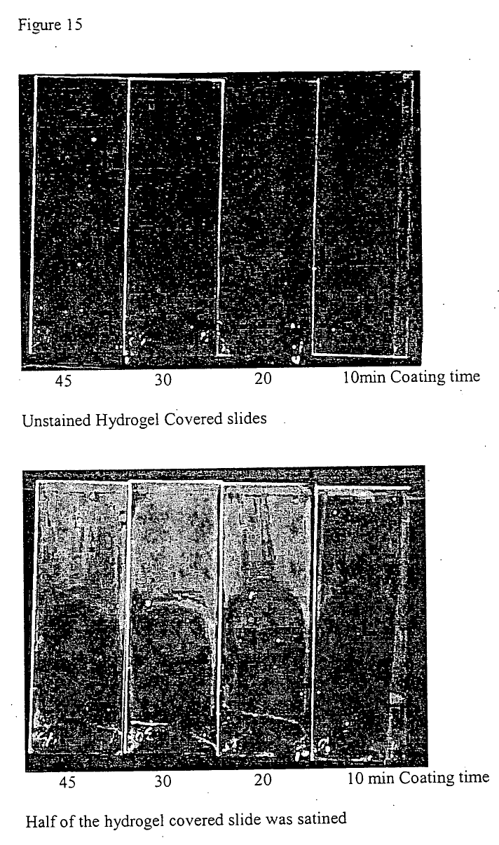

FIG. 15 depicts hydrogel covered slides. The slides were coated for various lengths of time (e.g., 10, 20, 30 and 45 minutes). The bottom set of hydrogel coated slides were stained with N-(4-(6-dimethylamino-2-benzofuranyl)phenylisothiocyanate on half of the slide. It can be observed that as coating time progresses the intensity of the fluorescence increases and becomes more homogeneous, reaching a plateau at around 30 min.

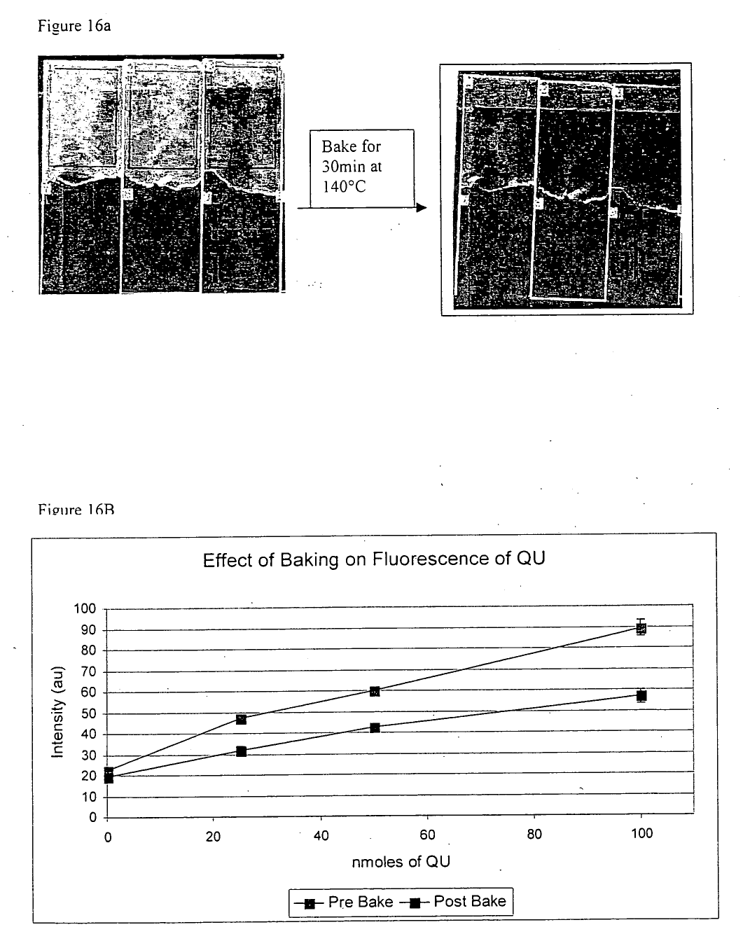

FIG. 16(a) depicts the before and after baking view of a set of slides coated with QU. The fluorescent signal of the set of slides is dramatically reduced after baking for 30 minutes at 140° C.

FIG. 16(b) graphically depicts the pre and post-baking effects of the fluorescent signal of QU coated slides at various concentrations (25, 50 and 100 nmoles of QU). After baking, the fluorescent signal decreased in all three groups. Biomolecules can be immobilized on a variety of solid surfaces, for a number of known applications, including for creating combinatorial complex carbohydrate arrays, for DNA and RNA oligomer synthesis, for separation of desired target nucleic acids from mixtures of nucleic acids including RNA; conducting sequence-specific hybridizations to detect desired genetic targets (DNA or RNA); creating affinity columns for mRNA isolation; quantification and purification of PCR reactions; characterization of nucleic acids by AFM and STM; for sequence determination of unknown DNAs, such as the human genome, etc. A number of methods have been employed to attach biomolecules to substrates. There are numerous patents and patent applications, which describe arrays of oligonucleotides and methods for their fabrication, and a variety of substrates for DNA immobilization, including polymeric membranes (nylon, nitrocellulose), magnetic particles, mica, glass or silica, gold, cellulose, and polystyrene, etc. They include: U.S. Pat. Nos. 5,077,210; 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,895; 5,624,711; 5,639,603; 5,658,734; 5,677,126; 5,688,642; 5,700,637; 5,744,305; 5,760,130; 5,837,832; 5,843,655; 5,861,242; 5,874,974; 5,885,837; 5,919,626; PCT/US98/26245; WO 93/17126; WO 95/11995; WO 95/35505; EP 742 287; and EP 799 897. There are numerous patents and patent applications describing methods of using arrays in various applications, they include: U.S. Pat. Nos. 5,143,854; 5,288,644; 5,324,633; 5,432,049; 5,470,710; 5,492,806; 5,510,270; 5,525,464; 5,547,839; 5,580,732; 5,661,028; 5,848,659; 5,874,219; WO 95/21265; WO 96/31622; WO 97/10365; WO 97/27317; EP 373203; and EP 785 280. The techniques and uses in these documents are all applicable herein.

The substrates to be modified for use in the methods and products of the present invention include materials, which have or can be modified to have surface reactive (e.g., hydroxyl) groups, which can react with a homogeneous mixture of a fluorescent-tagged compound and a chemically functional compound to which other chemical moieties can be bound. Suitable substrates are preferably inorganic materials, including but not limited to silicon, glass, silica, diamond, quartz, alumina, silicon nitride, platinum, gold, aluminum, tungsten, titanium, various other metals and various other ceramics. Alternatively, polymeric materials such as polyesters, polyamides, polyimides, acrylics, polyethers, polysulfones, fluoropolymers, etc. may be used as suitable organic substrates. The substrate used may be provided in any suitable form, such as slides, wafers, fibers, beads, particles, strands, precipitates, gels, sheets, tubing, spheres, containers, capillaries, pads, slices, films, plates, slides, etc. The substrate may have any convenient shape, such as that of a disc, square, sphere, circle, etc. The support can further be fashioned as a bead, dipstick, test tube, pin, membrane, channel, capillary tube, column, or as an array of pins or glass fibers.

Although the substrate may be made of a variety of either flexible or rigid, glass or plastic solid supports, glass is the preferred solid substrate. Additionally, the substrate may also be a coverslip, a capillary tube, a glass bead, a channel, a glass plate, a quartz wafer, a nylon or nitrocellulose membrane or a silicon wafer. The solid support can also be plastic, preferably in the form of a 96-well plate or 384-well plate. Preferably, the plastic support is a form of polystyrene plastic.

As mentioned above, an array can be present on either a flexible or rigid substrate. A flexible substrate is capable of being bent, folded or similarly manipulated without breakage. Examples of solid materials which are flexible solid supports with respect to the present invention include membranes, e.g., nylon, flexible plastic films, and the like. By “rigid” is meant that the support is solid and does not readily bend, i.e., the support is not flexible. As such, the rigid substrates for use in bioarrays are sufficient to provide physical support and structure to the associated biomolecules such as oligonucleotides and/or polynucleotides present thereon under the assay conditions in which the array is employed, particularly under high throughput handling conditions.

The substrate and its surface are also chosen to provide appropriate optical characteristics. In a preferred embodiment, the substrate is a low self-fluorescent glass, or a pure SiO₂ glass, most preferably, a low self-fluorescent borosilicate or soda-lime glass. In another preferred embodiment, the substrate is a gold-coated substrate such as gold-coated ceramic, gold-coated glass-ceramic or a gold coated polymeric substrate, most preferably a gold coated glass. Other suitable substrates include those disclosed in U.S. 20020044893A1; U.S. Pat. No. 6,127,129; EP 858 616; and U.S. Pat. No. 6,146,767. In addition, the substrate may also be a SiO₂ coated substrate or polymer such as (poly) tetrafluoroethylene, (poly)vinylidenedifluoride, polystyrene, polycarbonate, etc., or combinations thereof. If the chosen substrate is glass, it is often desirable to clean the glass substrate prior to coating with the homogenous mixture of a fluorescently labeled compound and a chemically functional compound. Suitable cleaning can be achieved according to conventional glass cleaning protocols.

The fluorescent-labeled compound and chemically functional compound are preferably fully miscible, and when mixed, coat a glass substrate in a random, homogeneous manner. This is because the uniformity of the chemically functional coating is being inferred by the uniformity of the fluorescent-labeled compound coating. The fluorescent-labeled compound preferably does not fluoresce or otherwise be detectable in a region that is of general importance for the end application, or in any other way significantly affect the ultimate end use of the functional compound.

For instance, the quality control method of the invention can be used for assessing the quality of coated substrates intended for nucleic acid (e.g., DNA), tissue, carbohydrate and protein arraying applications. Fluorescent characterization is the method of choice for nucleic acid and protein microarraying, and the most common dyes used for nucleic acid and protein microarraying (i.e., Fluor X™, Cy3™, and Cy5™ chemical dyes) have absorption maxima at 494 nm, 523 nm and 635 nm, and fluorescent emission at wavelengths ranging from 475-675 nm, 540-610 nm and 630-710 nm, respectively (see FIG. 3). Many other visible dyes may also be used (e.g., Texas Red, Bodipy, etc.). Therefore, the fluorescent-labeled entity, i.e., silane or alkanethiol, is the minor constituent of the homogenous coating mixture and is selected to impart optical activity to the substrate surface in a region that will not conflict with a region used for microarray experiments. Detection of the optical activity provides the quality control of substrates for microarraying applications. The fluorescent-labeled entity should preferably absorb in the UV and fluoresce in the “blue” region (to avoid high background fluorescence during a microarraying experiment). Various chemical dyes have been identified that exhibit such UV absorption and “blue” emission (see FIG. 4). Preferred fluorescent labeled compounds include compounds such as (4-methylcoumarinyl)-N-[3-triethoxysilylpropyl]carbonate; ([N-triethoxysilylpropyl]dansylamide; 4-methyl-3-trimthylsiloxycoumarin; 3-(2,4-dinitrophenylamino)propyltriethoxysilane; triethoxysilylpropylquinineurethane; and 3-(2,4-dinitrophenylamino)propyltriethoxysilane. Preferred fluorescent compounds which may be coupled to silanes include compounds such as LysoTracker Blue DND-22, anilinonaphthalene-8-sulfonic acid, 7-amino-4-methylcoumarin, and 6,8-difuoro-7-hydroxy-4-methylcoumarin. Any fluorescent labeled compound which does not fluoresce in a region that will interfere with the end assay applications, is useful. Of course, IR dyes are also contemplated.

In a preferred embodiment, the fluorescent portion of the fluorescent-labeled compound molecule is approximately the same molecular weight and size as the end group on the primary chemically functional compound ensuring that the two are miscible and that they coat the glass in a random, homogeneous manner. A pictorial representation of this aspect is shown in FIG. 5. The fluorescent-labeled compound has a sufficiently high extinction coefficient and quantum yield such that adequate optical fluorescence is obtained with low doping concentrations (<25%, preferably <10% and most preferably <0.1%).

In a preferred embodiment, the fluorescent activity of the fluorescent-labeled compound decays as a function of time. Alternatively, the fluorescent activity of the fluorescent-labeled compound may decay as a function of heat treatment. (See FIG. 16.) In yet another embodiment, the fluorescent activity of the fluorescent-labeled compound decays when subjected to high intensity ultraviolet light. (See FIG. 6.) These embodiments provide adequate fluorescence for quality control immediately after production, yet since the fluorescent activity of the compound degrades over time or when subjected e.g., to heat or photo-bleaching, there is greatly lessened or no chance of background fluorescence once the coated substrate is used by a customer in an end application (e.g., a microarray experiment). Furthermore, since the fluorescent compound decays before the final end use, the selection of a suitable dopant is greatly expanded to include dopants which fluoresce at the wavelengths used during a microarray experiment.

One method of forming the fluorescent labeled compound is to react a portion of a chemically functional molecule with a fluorophore. For example, a reaction dependent fluorophore could be conjugated to the amine group of a DETA molecule (i.e., trimethoxysilylpropyl-diethylenetriamine). The fluorescent laden molecules thus formed are then mixed with non-reacted DETA molecules to create a homogenous mixture of a labeled compound and a chemically functional compound.

The chemically functional compound, to which other chemical moieties can be bound and the labeled compound, can bind to the substrate, e.g., by condensation reactions or Au—S bonding. The chemically functional compound is the major constituent of the homogenous coating mixture and is selected to impart functionality to the glass surface (i.e., primary, secondary or tertiary amines, aldehyde, carboxylate, cyanate, epoxide, ester, ether, chloro, bromo iodo, ketone, vinyl (alkyl), acrylate, ethylene glycol, fluoro, hydroxy, isocyanate, isothiocyanate, NHS ester, thiol (mercaptan, sulfhydryl), etc. Such compounds are well known. Such functionality is useful for many biological and industrial applications. In particular, amino and epoxy silane coated substrates are commonly used for preparing DNA and protein microarrays. Preferred chemically functional compounds include alkanethiols and a wide variety of silanes, preferably epoxysilanes such as epoxycyclohexyl ethyltrimethoxysilane or glycidoxyproply trimethoxysilane, and most preferably aminosilanes such as aminopropyl-trialkoxysilane, aminobutyldimethylmethoxysilane, and multiaminosilanes having more than one amine group. Suitable chemically functional compounds for use in the homogenous coating mixture may be, for example, multiaminoalkyl monoalkoxysilane, multiamino-alkyl dialkoxy silane, and/or a multiaminoalkyl trialkoxysilane. Also suitable are multiaminoorganosilanes such as trimethoxysilylpropyl-diethylenetriamine (DETA), N-(2-aminoethyl)-3-aminopropyltrimethoxysilane (EDA), and/or (aminoethyl aminomethyl) phenethyltrimethoxysilane (PEDA). Also suitable are hydrogels, which are polymer networks capable of swelling in water. Typical hydrogels are derived from carbohydrates (chitosan, alginates, hyaluronic acid, etc.), proteins (e.g., collagen), and synthetic polymers, the most predominant ones being polyethylene glycols, nitrocellulose, polyurethane etc. There are numerous methods of producing hydrogels, which would be suitable for use as the chemically functional compound of the present invention. See for example Hennink W E Adv. Drug Deliv. Rev. 54:13-36 (2000) and Gehrke S F NY Acad. Sci. 831:179-207 (1997).

Coating of the homogeneous mixture of a fluorescent-tagged compound and a chemically functional compound can be performed directly onto the substrate surface, e.g., glass, an optionally SiO₂ coated substrate, an optionally acid leached substrate (to form SiO₂), etc. Such modification layers, when present, will generally range in thickness from a monomolecular thickness (typically <5 nm) to about several hundred microns.

The coatings of this invention can be continuous or discontinuous patterned coatings. Often the coating layer will be a monolayer. A monolayer coating is defined herein as an organic, inorganic or organometallic film that is formed on a substrate surface, whereby the film thickness is similar to the molecular size of the coating precursor. For example, a monolayer silane coating on glass typically has a thickness of <5 nm, because a uniform film of a silane molecules in formed on the glass surface, and most silane precursor molecules have a length of <5 nm. (See FIG. 9.) The use of self-assembled monolayers (SAMs) on surfaces for binding and detection of biological molecules has recently been explored. See for example WO98/20162; PCT US98/12430; PCT US98/12082; PCT US99/01705; PCT/US99/21683; PCT/US99/10104; PCT/US99/01703; PCT/US00/31233; U.S. Pat. Nos. 5,620,850; 6,197,515; 6,013,459; 6,013,170; and 6,065,573; and references cited therein.

Multilayer coatings are also contemplated. A multilayer coating is defined herein as an organic, inorganic or organometallic film that is formed on a substrate surface, whereby the film thickness is some integer multiple of the molecular size of the starting precursor. (See FIG. 10.)

A typical protocol for coating using a homogeneous mixture of a fluorescent-labeled compound and a chemically functional compound can be accomplished by shaking a clean substrate in the coating composition (0.01 to 100 wt % homogenous coating mixture of the coating molecules (e.g., silanes) and residual solvents) solution for 15 min. Preferably the homogenous coating mixture comprises from 1-10 wt % and most preferably from 4 to 6% of the coating molecules (e.g., silanes). The coating mixture can contain 5 to 99.9 wt % of H₂O and 5 to 99.9 wt % of an organic solvent such as acetone, toluene, isopropanol, methanol, ether or ethanol. Acids or bases may be used to adjust pH in an aqueous solution, but the coating solution is generally maintained at a pH of 1-14 and most preferably at a pH of 9-11 for glass substrate coating applications. After dip coating, the substrates are then shaken in ultra-pure, low residue methanol, ethanol and/or isopropanol again for about 0.1 to 24 hours and rinsed well with double distilled H₂O for about 0.1 to 24 hours. After rinsing the substrates are spin dried for about 5 min. at 1000 rpm (Class-100 clean room is optional) and heat-treated at a temperature of 25 to 250° C. for 0.1 to 24 hours, preferably at a temperature of 100-140° C., and most preferably at a temperature of 110-130° C. (Class-100 clean room is optional). The coated substrates can be stored in a vacuum desiccator. Coatings can also be achieved through thermal chemical vapor deposition (T-CVD). During T-CVD a homogeneous mixture (i.e., chemically functional and labeled compound) of coating molecules is evaporated into a chamber that contains clear glass substrates. The coating molecules (e.g., silanes) adsorb onto the clean glass substrates and subsequently form covalent bonds through condensation and/or Au—S bonding. This bonding can be accelerated through the use of heat. T-CVD can be conducted at temperatures of 25-250° C. but most preferably at temperatures of 70-150° C.

Each substrate coated with a mixture of chemically functional and fluorescent-labeled compounds can be assessed readily and non-destructively for quality using fluorescence, or other label detective method. There are numerous apparatus available for inspecting the coated substrates of the present invention. In one such apparatus, the coated substrate is illuminated with light suitable to excite the fluorescent compound and the flour emission is captured by a detector positioned above the substrate. The detector cooperates with processing means to determine the coating uniformity. The system permits accurate determinations of coating uniformity even when the coated substrate surface has irregularities. The system may be employed above a moving surface and may include mirrors and fiber optic components. A suitable conventional lens for focusing the returned light and a filter for excluding extraneous returned light from the detector may also be employed.

In another apparatus for inspecting the coated substrates of the present invention, ultraviolet radiation is projected onto the coated surface of the substrate; fluorescence emitted due to excitation is detected by a high-sensitivity camera (i.e., TV or CCD); the signal representing the intensity of detected fluorescence is amplified; and the quantity of coating is derived from the intensity of the amplified signal, which is processed to correct the linear relation between the quantity of coating and the intensity of fluorescence. This system enables accurate measurement of uniformity of coating film on a coated substrate in an on-line system. In a preferred inspection method the coated substrates are simply placed on a conveyer belt that introduces the samples into a “light box” which imparts the appropriate energy and intensity of light onto the coating to induce fluorescence. The fluorescence is then characterized using a video camera, and an operator decides whether uniform coatings were prepared based on the image displayed on a video screen. A pictorial representation of such a 100% non-destructive quality control process is shown in FIG. 2. In another embodiment, this same inspection procedure is accomplished using a commercially available fluorimeter. It is contemplated that any of the above quality inspection procedures can be performed by the manufacturer or as an added measure of quality control, the consumer can also perform inspection.

The quality controlled coated surfaces thus obtained are useful for attaching molecules, e.g., “biomolecules” such as cells, tissues, proteins, nucleic acids, lipids, sugars, carbohydrates, polysaccharides, RNAs, DNAs and derivatives thereof, as well as small molecules such as typical drugs. Typically, small molecules are of a nonpolymeric nature and include, but are not limited to organic or inorganic compounds (e.g., including heteroorganic and organometallic compounds) having a molecular weight less than about 10,000 grams per mole, preferably organic or inorganic compounds having a molecular weight less than about 5,000 grams per mole, and more preferably organic or inorganic compounds having a molecular weight less than about 1,000 grams per mole. A nucleic acid is a covalently linked sequence of nucleotides and includes “polynucleotides,” a nucleic acid containing a sequence that is greater than about 100 nucleotides in length; oligonucleotides, a short polynucleotide or a portion of a polynucleotide; and SNPs (single nucleotide polymorphisms) which are variations from the most frequently occurring base at a particular nucleic acid position. As used herein, the term “target nucleic acid” or “nucleic acid target” refers to a particular nucleic acid sequence of interest. Thus, the “target” can exist in the presence of other nucleic acid molecules or within a larger nucleic acid molecule. Among the proteins, are included any polyaminoacid chain, peptides, protein fragments and different types of proteins (e.g. structural, membrane, enzymes, antigens, antibodies, ligands, receptors) produced naturally or recombinantly; as well as the derivatives of these compounds, etc.

All RNAs and DNAs are included, e.g., alpha-, beta-derivatives as well as aminothiol derivatives and mixed compounds such as PNAs. Mixed compounds such as glycoproteins, glycopeptides and lipopolysaccharides for example, or alternatively other elements such as viruses, cells, or chemical compounds such as biotin, can also be attached.

Thus, in general, the fluorescently-labeled coated substrates of the present invention can be used in processes for detecting and/or assaying a molecule with biological activity in a sample, wherein a coated substrate as described above, on which a molecule with biological activity capable of recognizing the sample molecule (e.g. probe) becomes attached, is used, and the detection or assay is carried out using a reagent, fluorescent or otherwise, which detects the presence of the attached molecule. As mentioned, if the detection reagent chosen is fluorescent, it is selected so as to fluoresce in a region of the spectrum that is different than the fluorescent-labeled dopant used in the homogenous coating mixture. Thus, the optical activity used to characterize coating uniformity will not interfere with the optical activity of fluorescent agents attached to sample biomolecules. The activity of the biomolecule may be maintained after immobilization to the surface. For example, immobilized DNA or RNA probes may retain their ability to hybridize to a complementary DNA or RNA molecule in a sequence-specific manner, or to function as primers for nucleic acid amplification techniques.

Pin spotting and ink jet printing are the most common techniques used to place small volumes (spots) of solution, which contain known probes, onto a solid support. An ideal solid support for microarray applications would have zero self-fluorescence in the spectral region used for assaying, and would form a strong chemical (covalent) bond with the probes that are pin spotted or ink jet printed onto the surface. After covalent bonding is achieved, the probe/coated substrate interactions should be strong enough to survive washing with mild detergents and/or immersion in boiling H₂O. Such strong covalent bonding is desirable for DNA microarray applications, since it is often vital that the probes are immobilized on the solid support, and that they remain immobilized at a known location after various hybridization and cleaning steps.

With an automated delivery system, such as a Hamilton robot (e.g., Hamilton 2200 pipeting robot (Hamilton, Inc., Reno, Nev.)) or ink-jet printing method, it is possible to form a complex array of probes (e.g., DNA and/or oligonucleotide probes) on a solid support, in particular onto fluorescently labeled coated solid substrates. Such methods can deliver nano to pico-liter size droplets with sub-millimeter spacing. Because the aqueous droplets are well defined on such a hydrophobic surface, it is possible to create an array with a high density of probes (e.g., DNA and/or oligonucleotide probes). Thus, it is possible to create arrays having greater than about 10,000 probe droplets/cm². Such arrays can be assembled through the use of a robotic liquid dispenser (such as an ink-jet printing device controlled by a piezoelectric droplet generator). Methods and apparatus for dispensing small amount of fluids using such ink-jet printing techniques and piezoelectric ink-jet depositions have been previously described by Wallace et al. (U.S. Pat. No. 4,812,856), Hayes et al. (U.S. Pat. No. 5,053,100), both of which are herein incorporated by reference in their entirety. The array can also be created by means of a “gene pen”. A “gene pen” refers to a mechanical apparatus comprising a reservoir for a reagent solution connected to a printing tip. The printing tip further comprises a means for mechanically controlling the solution flow. A multiplicity of “gene pens” or printing tips may be tightly clustered together into an array, with each tip connected to a separate reagent reservoir or discrete “gene pens” may be contained in an indexing turntable and printed individually. Alternatively, the array can be created with a manual delivery system, such as a pipetman. Because these arrays are created with a manual delivery system, these arrays will generally not be as complex as those created with an automated delivery system. Arrays created with a manual delivery system will typically be spaced further apart. Preferably, arrays created with a manual delivery system will be created in a 96-well or 384-well plate or larger.

Another preferred use of the fluorescently-labeled modified substrates of the present invention is for creating carbohydrate arrays, which can be exploited in a variety of ways, including, but not limited to, (i) identification of complex carbohydrate drugs; (ii) identification of complex carbohydrate associated receptors or proteins as potential new carbohydrate related targets for drug therapy; (iii) identification of biologically-active complex carbohydrates; (iv) identification of specific complex structural carbohydrate elements as potential new targets for drug therapy; (v) identification of the active sites of known complex carbohydrate structures; (vi) identification of new glycomarkers in complex carbohydrate structures; and (vii) detection of antibodies formed against a cancer-related glyco-epitope or other disease related glycoantigens.

Another preferred use of the fluorescently-labeled modified substrates of the present invention is for creating DNA microarrays. Arrays are generally comprised of known, single-stranded nucleic acid fragments (e.g., SNP's; probes) that are attached to a solid support in known locations. The DNA microarray is generally used as a tool for identifying unknown, single-stranded cDNA fragments (targets) that exist in a buffered solution. These targets are often formed during expression analysis or SNP detection experiments, and are tagged with a fluorescent dye for identification purposes.

The unknown targets are identified using a hybridization experiment, whereby a DNA microarray (containing the probes) and a buffered solution (containing the targets) are combined. When combined, complimentary probes and targets hybridize, forming chemically stable, hydrogen bonded double stranded DNA at specific locations on the microarray. The hybridized microarray is then rinsed and analyzed with a fluorescent scanner to semi-quantitatively determine the identity and concentration of target probes produced during the expression analysis or SNP detection experiment. The fluorescent scanner used to analyze the hybridized microarray scans in a region other than the 300-550 nm wavelength; so as not to detect the fluorescence of the homogenous fluorescently labeled substrate coating. In a typical DNA microarray experiment a DNA chip containing known DNA fragments (probes) at known locations is introduced to a solution containing fluorescently tagged, unknown DNA (targets), hybridization occurs between complimentary DNA fragments, and a scanner is then used to determine the identity and quantity of unknown DNA.

Although fluorescence is a preferred method, any method of determining the analytical output can be used. A label, tag, radioisotope, molecule, or any substance, which emits a detectable signal or is capable of generating such a signal, e.g., luminescence enzyme, or any of the variety of known signaling entities are useful.

In a preferred embodiment, the analytical output is obtained by fluorescent spectroscopic methods using fluorescent dyes that do not fluoresce in the same region as the homogenous fluorescently-labeled substrate coating. Use of a wide variety of fluorescence detection methods is contemplated. The fluor (fluorescent dye) can be coupled directly to the pyrimidine or purine ring of the nucleotides of the probe (Ried, T. et al., Proc. Natl. Acad. Sci. (U.S.A.) 89:1388-1392 (1992), and; U.S. Pat. Nos. 4,687,732; 4,711,955; 5,328,824; and 5,449,767, each herein incorporated by reference). Alternatively, the fluor may be indirectly coupled to the nucleotide, as for example, by conjugating the fluor to a ligand capable of binding to a modified nucleotide residue.

The most common fluorescent dyes used for DNA microarray applications are Cy3™ and Cy5™. The Cy3™ absorption and emission windows are centered at 550 nm and 570 nm, respectively, while the Cy5™ absorption and emission windows are centered at 649 nm and 670 nm, respectively. FIG. 3 depicts plots of the emission spectra for FluorX™, Cy3™ and Cy5™. Although Cy3™ and Cy5™ are the most common fluors for detecting assay activity, other fluors can be used such as 4′-6-diamidino 2-phenyl indole (DAPI), fluorescein (FITC), and the new generation cyanine dyes Cy3.5, Cy5.5 and Cy7. Of these, Cy3, Cy3.5, Cy5 and Cy7 are particularly preferred. The absorption and emission maxima for the respective fluors are: DAPI (absorption maximum: 350 nm; emission maximum: 456 nm), FITC (absorption maximum: 490 nm; emission maximum: 520 nm), Cy3 (absorption maximum: 554 nm; emission maximum: 568 nm), Cy3.5 (absorption maximum: 581 nm; emission maximum: 588 nm), Cy5 (absorption maximum: 652 nm; emission maximum: 672 nm), Cy7 (absorption maximum: 755 nm; emission maximum: 778 nm). Complete properties of selected fluorescent labeling reagents are provided by Waggoner, A. (Methods in Enzymology 246:362-373 (1995) herein incorporated by reference). In light of the above, it is readily apparent that other fluorophores having adequate spectral resolution can alternatively be employed in accordance with the methods of the present invention.

The disclosures of U.S. Pat. Nos. 5,348,853; 5,119,801; 5,312,728; 5,962,233; 5,945,283; 5,876,930; 5,723,591; 5,691,146; and 5,866,336 disclosing fluorophore labeled oligonucleotides are incorporated herein by reference. Guidance for making fluorescent intensity measurements and for relating them to quantities of analytes is available in the literature relating to chemical and molecular analysis, e.g. Guilbault, editor, Practical Fluorescence, Second Edition (Marcel Dekker, New York, 1990); Pesce et al, editors, Fluorescence Spectroscopy (Marcel Dekker, New York, 1971); White et al, Fluorescence Analysis: A Practical Approach (Marcel Dekker, New York, 1970); and the like. Substrates of the present invention have numerous uses including “adhesive scaffolds,” upon which tissue engineering could be conducted; in DNA hybridization analysis to detect or identify a genetic target such as a specific nucleic acid sequence, microorganism, genetic disorder etc.; to obtain patterns or arrays of nucleic acids that may be non-covalently or covalently bound to the substrate, etc. Handling conditions and reagents should be chosen such that they are not destructive to the underlying coating or to the substrate, and to promote maximum density of biomolecules attached to the substrate.

These specific examples are not intended to limit the scope of the invention described in this application. Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius; and, unless otherwise indicated, all parts and percentages are by weight.

### EXAMPLE 1

### Background Self-Fluorescence Study

TABLE 1

| borosilicate 1 | | borosilicate 2 | | borosilicate 3 | | soda lime silicate 1 | |
|---|---|---|---|---|---|---|---|
| SiO₂ | 58-85, | SiO₂ | 40-60 | SiO₂ | 60-70 | SiO₂ | 65-75 |
| B₂O₃ | 7-15, | B₂O₃ | 10-20 | B₂O₃ | 5-10 | Na₂O | 5-15 |
| Al₂O₃ | 0-8, | Al₂O₃ | 8-20 | Al₂O₃ | 0.1-8 | K₂O | 5-15 |
| Na₂O | 0-15, | BaO | 20-30 | Na₂O | 0-8 | ZnO | 2-6 |
| K₂O | 0-8, | Na₂O | 0-5 | K₂O | 0-8 | TiO₂ | 0.1-5 |
| ZnO | 0-8, | K₂O | 0-5 | ZnO | 3-10 | BaO | 0.1-5 |
| CaO | 0-8, | ZnO | 0-7 | TiO₂ | 1-10 | CaO | 0-10 |
| MgO | 0-8, | CaO | 0-8 | CaO | 0-5 | MgO | 0-6 |
| As₂O₃ | 0-2, | MgO | 0-5 | MgO | 0-5 | PbO | 0-3 |
| Sb₂O₃ | 0-2. | As₂O₃ | 0-2 | As₂O₃ | 0-2 | Al₂O₃ | 0-3 |
| | | Sb₂O₃ | 0-2. | Sb₂O₃ | 0-2. | B₂O₃ | 0-5 |
| | | | | | | As₂O₃ | 0-2 |
| | | | | | | Sb₂O₃ | 0-2. |

The glass types-utilized for the self-fluorescence study were borosilicate 1, borosilicate 2, borosilicate 3, soda lime silicate and SiO₂. The first three glasses are multi-component oxide silicate glasses that are produced in a thin, flat format that is suitable for DNA microarray applications (see table 1). SiO₂ is an extremely pure glass produced by a chemical vapor deposition technique, which exhibits low self-fluorescence. SiO₂ must be cut, ground and polished to obtain a substrate suitable for DNA microarray applications. The self-fluorescence of commercially available Corning CMT GAP and TeleChem Arraylt slides were also measured for comparative purposes.

The various glasses described above were scanned for self-fluorescence before and after being coated with DETA, using a green (523 nm, for Cy3™ excitation) and red (635 nm, for Cy5™ excitation) laser using a GenePx 4000A fluorescent scanner. All background readings were scanned at 100% laser power and at constant sensitivity (photomultiplier tube gain; PMT=700), using an Axon GenePix Pro 3 scanner and software package. Fluorescence intensities for each of the coated glasses were calculated on a relative scale and normalized for thickness (i.e., the relative fluorescent value is divided by the thickness of the glass, nominal thickness of SiO₂=2.3 mm, borosilicate 1=1.2 mm, borosilicate 2=1.1 mm, and borosilicate 3=1.1 mm). There was no statistically significant difference for the self-fluorescence data obtained before and after coating with DETA, thus the glass substrate was primarily responsible for self-fluorescence.

### EXAMPLE II

### Mixtures of Fluorescent and Chemically Functional Silane Molecules

An appropriate fluorescent silane molecule was selected based on the excitation and emission spectra of the silane (characterized using a fluorimeter) and the excitation and emission spectra of the chemical dye(s) were used during the microarray experiment. The fluorescent silane was chosen so that there was no overlap between the emission spectra of the silane and the dye(s) used for target labeling during the microarray experiment. Once chosen, the fluorescent silane (i.e., N-(triethoxysilylpropyl)dansylamide) was mixed with a chemically functional silane (i.e., trimethoxysilylpropyl-diethylenetriamine or glycidoxyproply trimethoxysilane) in the proportions listed in Table II.

TABLE II
Typical Graded Concentration Series for Fluorescent/
Chemically Functional Silane Mixtures.

| Graded | | Chemically Functional |
|---|---|---|
| Concentration | Fluorescent Silane | Silane Concentration |
| Series | Concentration (mol %) | (mol %) |
| 1 | 25 | 75 |
| 2 | 10 | 90 |
| 3 | 1 | 99 |
| 4 | 0.1 | 99.9 |
| 5 | 0.01 | 99.99 |

Each mixed silane compositions shown in Table I was then coated onto clean borosilicate glass substrates by dip or thermal CVD coating. Once coated, the substrates were first scanned using the apparatus intended for conducting 100% non-destructive quality to control (see FIG. 2). The goal of the initial scanning experiment was to determine which concentrations provide sufficient fluorescence for the assessment of coating uniformity. Once this limit was determined, the slides were scanned a second time using a microarray scanner (i.e., an Axon, GSI Lumonix, Affymetrix or Applied Precision scanner). This second scanning experiment was used to unsure that the fluorescent-silane-doped coatings did not exhibit fluorescence under the excitation and emission conditions that will be used during the microarray experiment. The overall goal of this study was to choose the lowest possible fluorescent silane doping level that would allow for 100% non-destructive assessment of coating uniformity without deleteriously affecting the results from a subsequent microarray scanning assay.

After the acceptable concentrations of fluorescent and chemically functional silanes were determined, the mixed, coated substrates were assessed for biological performance. During this stage, the hybridization results obtained from mixed (i.e., 10% fluorescent, 90% chemically functional) silane coated substrates were compared with those obtained from substrates coated with 100% chemically functional silane. The substrates were coated with the mixed and 100% chemically functional silanes via thermal CVD and/or dip coating. After coating, the mixed and 100% chemically functional coated substrates were assessed for biological performance by conducting a DNA, protein or carbohydrate hybridization experiment. A typical DNA microarray hybridization experiment was used to compare the biological performance of mixed and 100% chemically functional coated substrates.

Microarrays were printed on all substrates using 60-base oligonucleotide probes and a commercially available printer (i.e., Genemachine, Omnigrid). After printing, the oligonucleotides were immobilized onto the coated substrate by UV crosslinking and then hybridized overnight with a dilute solution of 100% complimentary, Cy3-labeled, 60-base oligonucleotide targets. After hybridization, the microarrayed substrates were washed and scanned using a commercially available scanner (i.e., Axon, Affymetrix, GSI Lumonix) and average S/Bk values were calculated for the substrates coated with mixed and 100% functional silanes. There was no statistically significant difference (to a 90% confidence level) between the substrates coated with the mixed and 100% functional silanes.

### EXAMPLE III

### Dip Coating with a Silane Mixture Containing N-Triethoxysilylpropylquinineurethane (QU), a Fluorescent Silane

Clean slides were dip coated in solutions containing different molar percentages of N-triethoxysilylpropylquinineurethane (QU) and (3-trimethoxysilylpropyl)diethylenetriamine (DETA) under the following proportions:

| Slides | % DETA | % QU |
|---|---|---|
| 1 | 99 | 1 |
| 2 | 90 | 10 |
| 3 | 50 | 50 |
| 4 | 0 | 100 |

Slides were then observed by exciting the slide with a 365 nm UV light The difference was observed between the two extremes, 1/99% QU/DETA and 100% QU. A significant difference between the two was observed (See FIG. 14a). These slides were used as substrates for a DNA microarraying experiment. There was no significant difference between the slides. The values are the same as the averages observed for regular DETA coated slides in the same experiment (See FIG. 14b.)

### EXAMPLE V

### Reacting a Portion of Chemically Active Silane Solution with a Fluorophore Prior to Coating

An appropriate, reactive fluorophore molecule was selected based on the excitation and emission spectra obtained using a fluorimeter, and the excitation and emission spectra of the chemical dye(s) that will be used during the microarray experiment. The reactive fluorophores was chosen such that there was no overlap between the emission spectra of the fluorophore and the dye(s) used for target labeling during the microarray experiment. Once chosen, the fluorophore was reacted with a chemically functional silane (i.e., trimethoxysilylpropyl-diethylenetriamine or glycidoxyproply trimethoxysilane) in various proportions, as shown in Table III.

TABLE III
Typical Graded Concentration Series Used for Mixing a Reactive
Fluorophore with a Chemically Functional Silane Coating

| Graded | | Chemically Functional |
|---|---|---|
| Concentration | Reactive Fluorophore | Silane |
| Series | Concentration (mol %) | Concentration (mol %) |
| 1 | 25 | 75 |
| 2 | 10 | 90 |
| 3 | 1 | 99 |
| 4 | 0.1 | 99.9 |
| 5 | 0.01 | 99.99 |

The various solutions (Table III), were first characterized for optical activity using a fluorimeter to ensure that the reactive fluorophore was indeed reacted with some portion of the chemically functional silane molecules, and imparted optical activity upon the mixed coating solution. Once this reaction was confirmed, each mixed coating solution was coated onto clean borosilicate glass substrates. These coated substrates were then scanned using the apparatus intended for conducting 100 % non-destructive quality to control (see FIG. 2). The goal of this initial scanning experiment was to determine which fluorophore/chemically active silane mixture provides sufficient fluorescence for the assessment of coating uniformity. Once this limit was determined, the slides were scanned a second time using a microarray scanner (i.e., an Axon, GSI Lumonix, Affymetrix or Applied Precision scanner). This second scanning experiment was used to insure that the fluorophore-doped coatings did not exhibit fluorescence under the excitation and emission conditions that will be used during the microarray experiment. A goal of this study was to choose the lowest possible fluorophore doping level that would allow for 100% non-destructive assessment of coating uniformity without deleteriously affecting the results from a subsequent microarray scanning assay.

To obtain other fluorescent compounds it was possible to react molecules that were not fluorescent initially, but became fluorescent when reacted with a group on the coating molecule. An example of this is the compound OPA (o-phthaidialdehyde) which was not fluorescent until it reacted with an amine and formed a fluorescent compound.

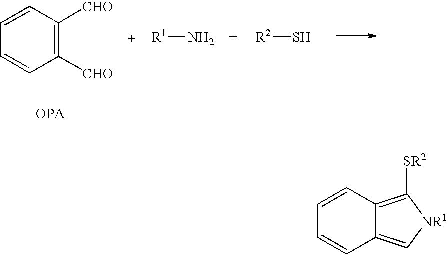

These types of reactions were also be used to modify the aminosilanes that coated the glass slide.

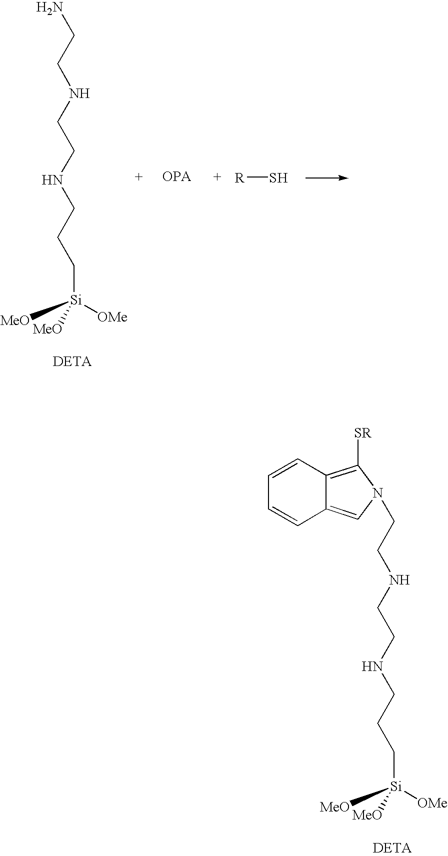

The fluorescent DETA was mixed with regular DETA and the mixture was coated onto the slides. The homogenous distribution of the fluorescent DETA allows control of the quality of the coating applied to the slide. The fact that different reactive compounds were used to produce the fluorescent derivative of different aminosilanes adds to the versatility of the method. Many compounds were used to produce these fluorescent coatings. Table III shows some of these compounds.

TABLE IV
List of amine reactive compounds used to derivatize
primary amine containing coating molecules.

| | Absorbance | Emission |
|---|---|---|
| Name of Compound | Wavelength (nm) | Wavelength (nm) |
| 9-anthracenepropionic acid | 366 | 414 |
| 2-anthracenesulfonyl chloride | 382 | 421 |
| N-(4-(6-dimethylamino-2- | 348 | 425 |
| benzofuranyl)phenylisothiocyanate | | |
| Fluorescamine | 380 | 464 |
| N-methylisatoic anhydride | 353 | 426 |
| naphthalene-2,3-dicarboxaldehyde | 419 | 493 |
| (NDA) | | |
| 1-pyreneisothiocyanate | 341 | 395 |
| o-phthaldialdehyde (OPA) | 334 | 455 |
| succinimidyl N-methylanthranilate | 368 | 437 |

NOTE:
The spectral characteristics described depend on various aspects of the measured compound including the solvent and the moiety on the reactive amine.

After the acceptable concentrations of fluorescent and chemically functional silanes were determined, substrates were coated with the appropriate fluorophore/chemically active silane mixture and assessed for biological performance. During this stage, the hybridization results obtained from fluorophore-doped coated substrates were compared with those obtained from substrates coated with 100% chemically functional silane. The substrates were coated via thermal CVD and/or dip coating. After coating, the fluorophore-doped and 100% chemically functional coated substrates are assessed for biological performance by conducting DNA, protein or carbohydrate hybridization experiments. A typical DNA microarray hybridization experiment was used to compare the biological performance of fluorophore-doped and 100% chemically functional coated substrates.

Microarrays were printed on all substrates using 60-base oligonucleotide probes and a commercially available printer (i.e., Genemachine, Omnigrid). After printing, the oligonucleotides were immobilized onto the coated substrates by UV crosslinking and then hybridized overnight with a dilute solution of 100% complimentary, Cy3-labeled, 60-base oligonucleotide targets. After hybridization, the microarrayed substrates were washed and scanned using a commercially available scanner (i.e., Axon, Affymetrix, GSI Lumonix) and average S/Bk values were calculated for the substrates coated with florophore-doped and 100% functional silanes. There was no statistically significant difference (to a 90% confidence level) between the substrates coated with the fluorophore-doped and 100% functional silanes.

### EXAMPLE V

### Fluorescent Staining of a Hydrogel

In a similar fashion the quality of a hydrogel coating was determined. The polymers contained in the hydrogel have amine moieties. These were reacted with the fluorophore generating compounds and the homogeneity of the distribution determined in a destructive manner. A non-destructive assay was also developed where some of the amine containing polymers of the hydrogel contain a fluorophore, and these were mixed in adequate proportions with non-derivatized amine polymers. The hydrogel was coated and the presence and homogeneity of the hydrogel coating was determined by exposure of the fluorescent compound to its excitation wavelength.

Another form of doing 100% QC in the case of hydrogels was to incorporate a fluorescent compound into the hydrogel coating solution that was not chemically bound to any of the constituents of the hydrogel. Once the coating was complete the fluorescent compound remained trapped in the gel matrix and homogeneity of the coating was determined by observing the fluorescence of the slide. One advantage in this case was that the slide was incubated in a bath that allowed the fluorescent compound to leach out, thereby removing the fluorescent compound before conducting a microarray. There was no interference in the assays the coated substrate was intended for.

In either experiment it was important to determine how the presence of the fluorescent compound affected the assay conditions of the hydrogel substrate. Comparisons were done with and without the fluorophore. If the flourophore was immobilized in the gel the slides were scanned under detection conditions to determine homogeneity of the coating as well as under the conditions typically used for microarray experiments to determine if the fluorescent molecule interfered with these conditions.

The biological performance of hydrogels with and without the fluorophores was compared to determine if these compounds had any effect on the protein assays performed. These tests were also performed using DNA.

### EXAMPLE VI

### Labile Fluorescent Coating (Post Quality Control Heat Treatment)

Slides were coated with N-triethoxysilylpropylquinineurethane (QU) and scanned to determine the intensity of the signal. After scanning the slides were baked at 140° C. for 30 minutes. The baked slides were scanned again to determine the decrease in signal strength.

In FIG. 16a it can be seen that after baking the intensity of the fluorescent signal decreases. This is independent of concentration, and the fluorescence typically decreases by 30% (See FIG. 16b).

Therefore, after utilizing the fluorescent signal of N-triethoxysilylpropylquinineurethane (QU) for 100% non-destructive assessment of coating uniformity, the slides can be exposed to heat and the labile characteristics of N-triethoxysilylpropylquinineurethane (QU) can be used to reduce or degrade the fluorescent signal so that there is no interference in the regions used for microarray experiments.

### EXAMPLE VII

### Results

The detection limits of fluorescent silanes were determined and proved using the concept of the amino reactive fluorescent staining techniques. Examples of these results:
1. N-(triethoxysilylpropyl)Dansylamide was obtained from Gelest Inc. This fluorescent silane is a liquid that was further diluted to lower concentrations. These solutions were spotted on a glass slide and the fluorescence was observed using a system comprised of a UV light box for illumination at 365 nm and a CCD camera with a blue filter for detecting the signal. FIG. 12 shows the detection limit of the dansylamide (DA) using this system (Slide 1)

As can be seen in FIG. 12, the observable detection limit is between 0.1 and 1 nmol of DA. Of course the detection will also be dependent of the area that the DA covers, and a broader area will mean a decrease in signal. Slides 2, 3, and 4 of FIG. 12 show different concentrations of DA spread across a slide manually. 10 nmoles of the DA gives an observable signal that is above the background, while 1 nmol is the same as the background. This proves that the dansylamide can be detected and that this method is a valid one for 100% QC of the slides produced.
2. The reactive staining of slides coated with aminosilanes was also proven.

Fluorescamine (Molecular Probes, Inc.) was reacted with DETA coated slides and produced fluorescence. As can be seen in FIG. 13, fluorescence is produced in the DETA coated slides. The levels are above the background from the control slide and from those corresponding to untreated DETA slides. The reaction appeared to be very quick and the fluorescence reached its peak within 15 minutes.
3. The reactive staining of the hydrogel can be seen in FIG. 15. In this case the fluorescent compound was used to optimize the coating of the slide by determining the fluorescence at different times during the coating reaction. It can be observed that as coating time progresses the intensity of the fluorescence increase and become more homogeneous, reaching a plateau at around 30 min.

### EXAMPLE VIII

### Post Quality Control Photobleaching

Fluorescent compounds were exposed to UV light for extended periods of time and high intensity UV light. When a fluorescent compound is excited it emits light, returns to its original energy level and can be excited again: When the excitation is of very high intensity or for prolonged periods of time it loses its ability to return to its original state and cannot fluoresce if excited. This phenomenon is called photobleaching.

In this example slides were coated with different amounts of the fluorescent silane N-triethoxysilylpropylquinineurethane (QU). These slides were then exposed to a 350 nm UV light for extended periods of time and the fluorescent signal was measured at different time intervals. The graph in FIG. 6 shows that as the slide is exposed to more UV light the fluorescence signal decreases. The different concentrations level out in about 60 minutes becoming asymptotic to the signal obtained with uncoated glass. Therefore, after 100% quality control the fluorescent coated slides can be exposed to UV to reduce or degrade the fluorescent signal so that there is no interference in the regions used for microarray experiments.

The entire disclosure of all applications, patents and publications, cited above or below, is hereby incorporated by reference.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1-34. (canceled)

35. A non-destructive method of determining the uniformity of a coating on a substrate comprising:
detecting the presence and locations of a labeled compound on a substrate which has been coated with a homogenous coating mixture comprising
a labeled compound and
a chemically functional compound
other molecules being chemically bondable to said functional compound when in said coating.

36. A method of claim 35 wherein the label is a fluorescent moiety and said detecting comprises subjecting the coated substrate to radiant energy to cause the fluorescent labeled compound to emit in the 300-520 nm wavelength range.

37. A method of claim 35 wherein the label is a fluorescent moiety and said detecting comprises subjecting the coated substrate to IR energy to cause the fluorescent labeled compound to emit in the >700 nm wavelength range.

38. A method as in claim 36, wherein said fluorescent labeled compound is 100%-1% by weight of the homogenous coating mixture.

39. A method as in claim 36, wherein said chemically functional compound is an alkanethiol.

40. A method as in claim 35, wherein said chemically functional compound is an alkylalkoxysilane.

41. A method as in claim 36, wherein said substrate comprises chemical moieties for conducting a bioassay and said fluorescent labeled compound does not affect the biofunctional properties of the coated substrate.

42. A method as in claim 36, wherein said substrate is glass.

43. The method of claim 36, wherein said homogenous coating mixture is applied to a gold-coated substrate.

44. The method of claim 43, whereby the substrate is gold-coated glass, gold coated ceramic, gold-coated glass-ceramic or a gold-coated polymeric substrate.

45. A method as in claim 36, further comprising examining the coated substrate for uniformity of fluorescence in the 300 to 550 nm wavelength region.

46. The method of claim 36, wherein the fluorescent labeled compound is
N-(Triethoxysilylpropyl) dansylamide,
5-Dimethylamino-N-(3-Triethoxysilylpropyl)napthalene-1-sulfonamide,
N-Triethoxysilylpropylquinineurethane,
3-(2,4-Dinitrophenylamino)propyltriethoxysilane, or mixtures thereof.

47 The method of claim 36, wherein the substrate is a substrate for DNA, protein function, protein capture, carbohydrate, or tissue microarray applications.

48. The method of claim 40, wherein the chemically functional coating is an alkylalkoxysilane monolayer.

49. The method of claim 36, wherein the chemically functional coating is an alkanethiol monolayer.

50. A method according to claim 36, wherein the homogenous coating mixture is applied to the substrate by chemical vapor deposition, sputtering, dip coating, ion beam deposition, flame hydrolysis deposition, laser pyrolysis deposition, liquid phase deposition, electron beam deposition, plasma arc deposition or flash evaporation deposition.

51. A method according to claim 36, wherein the substrate is a low self-fluorescent multi-oxide component glass.

52. A method according to claim 51, wherein the substrate is a low self-fluorescent borosilicate or soda lime silicate glass.

53. A method according to claim 36, wherein the chemically functional coating is a multiaminoalkylmonoalkoxy silane, multiaminoalkyldialkoxy silane, and/or a multiaminoalkyltrialkoxy silane.

54. A method according to claim 53, wherein the multiamino organosilane is trimethoxysi lylpropyl-diethylenetriamine(DETA), N-(2-aminoethyl)-3-aminopropyltrimethoxysilane(EDA), (aminoethyl aminomethyl) phenethyltrimethoxysilane (PEDA), or mixtures thereof.

55. A method according to claim 42, wherein said glass comprises, in % by weight on an oxide basis:

| | |
|---|---|
| SiO₂ | 58-85 |
| B₂O₃ | 7-15 |
| Al₂O₃ | 0-8 |
| Na₂O | 0-15 |
| K₂O | 0-8 |
| ZnO | 0-8 |
| CaO | 0-8 |
| MgO | 0-8 |
| As₂O₃ | 0-2 |
| Sb₂O₃ | 0-2. |

56. A method according to claim 42, wherein said glass comprises, in % by weight on an oxide basis:

| | |
|---|---|
| SiO₂ | 40-60 |
| B₂O₃ | 10-20 |
| Al₂O₃ | 8-20 |
| BaO | 20-30 |
| Na₂O | 0-5 |
| K₂O | 0-5 |
| ZnO | 0-7 |
| CaO | 0-8 |
| MgO | 0-5 |
| As₂O₃ | 0-2 |
| Sb₂O₃ | 0-2. |

57. A method according to claim 42, wherein said glass comprises, in % by weight on an oxide basis:

| | |
|---|---|
| SiO₂ | 60-70 |
| B₂O₃ | 5-10 |
| Al₂O₃ | 0.1-8 |
| Na₂O | 0-8 |
| K₂O | 0-8 |
| ZnO | 3-10 |
| TiO₂ | 1-10 |
| CaO | 0-5 |
| MgO | 0-5 |
| As₂O₃ | 0-2 |
| Sb₂O₃ | 0-2. |

58. A method according to claim 42, wherein said glass comprises, in % by weight on an oxide basis:

| | |
|---|---|
| SiO₂ | 65-75 |
| Na₂O | 5-15 |
| K₂O | 5-15 |
| ZnO | 2-6 |
| TiO₂ | 0.1-5 |
| BaO | 0.1-5 |
| CaO | 0-10 |
| MgO | 0-6 |
| PbO | 0-3 |
| Al₂O₃ | 0-3 |
| B₂O₃ | 0-5 |
| As₂O₃ | 0-2 |
| Sb₂O₃ | 0-2. |

59. An array of immobilized carbohydrate molecules comprising a plurality of carbohydrate molecules attached to a substrate, the uniformity of said molecules on said substrate having been determined by the method of claim 36.

60. (canceled)

61. A method of preparing a coated substrate whose coating uniformity can be nondestructively determined comprising:
coating the substrate with a homogenous coating mixture comprising
(a) a labeled compound and
(b) a chemically functional compound
other molecules being chemically bondable to said functional compound when in said coating.

62-78. (canceled)

79. A method of preparing a coated substrate for microarray applications comprising:
i) coating the substrate with a homogenous coating mixture comprising
a fluorescently labeled compound and
a chemically functional compound
other molecules being chemically bondable to said functional compound when in said coating,
ii) generating and detecting the fluorescent signal from said fluorescently labeled compound, and thereafter,
iii) reducing the fluorescent activity of the fluorescent labeled compound.

80. A method according to claim 79, wherein the fluorescent signal is detected to determine the uniformity of the homogenous coating mixture on a substrate.

81. A method according to claim 79, wherein the fluorescent activity of the fluorescent-labeled compound is reduced by photo bleaching.

82. A method according to claim 79, wherein the fluorescent activity of the fluorescent-labeled compound is reduced by heat treatment.

83. A method according to claim 79, wherein the activity of the fluorescent labeled compound is reduced to a level that does not interfere with optical measurements used for microarraying applications.

84. A method according to claim 79, wherein the substrate is a low self-fluorescent glass.

85. A method according to claim 79, wherein the glass is borosilicate or soda lime silicate glass.

86. A method according to claim 79, wherein the fluorescent labeled compound is derived from the reaction of a fluorescent dye and a second chemically functional compound.

87. A method according to claim 79, wherein the chemically functional compound is an organosilane.

88. The method of claim 79, wherein the substrate is for nucleic acid, protein function, protein capture, carbohydrate, small molecule, cell or tissue microarray applications.
